SEQUENCE LISTING (11 sequences)

SEQ ID NO: 1 - DNA, 42 bp, Artificial
  Tet-operon sequence
cgagtttaccactccctatcagtgatagagaaaagtgaaagt

SEQ ID NO: 2 - DNA, 60 bp, Artificial
  sense primer
cgacgcgttgcagagctcgttagagagataattagaattaatttgactgtaaacacaaag

SEQ ID NO: 3 - DNA, 42 bp, Artificial
  antisense sequence
cgggatccagaagaccacggtgtttcgtcctttccacaagat

SEQ ID NO: 4 - DNA, 64 bp, Artificial
  oligonucleotide
accgcaagctgaccctgaagttcttcaagagagaacttcagggtcagcttgctttttctcgagg

SEQ ID NO: 5 - DNA, 64 bp, Artificial
  oligonucleotide
gatccctcgagaaaaagcaagctgaccctgaagttctctcttgaagaacttcagggtcagcttg

SEQ ID NO: 6 - DNA, 36 bp, Artificial
  sense primer
cggaattcaccatgtctagactggacaagagcaaag

SEQ ID NO: 7 - DNA, 45 bp, Artificial
  antisense primer
cgggatcctgaagactacggtccgccgctttcgcactttagctgt

SEQ ID NO: 8 - DNA, 67 bp, Artificial
  oligonucleotide
accgaacctgttcgctctccccgctgcaacagcgggagccctactgacatcagcaccgtagtcttcg

SEQ ID NO: 9 - DNA, 67 bp, Artificial
  oligonucleotide
gatccgaagactacggtgctgatgtcagtagggctcccgctgttgcagcggggagagcgaacaggtt

SEQ ID NO: 10 - DNA, 39 bp, Artificial
  oligonucleotide
gatccgcaagctgaccctgaagttcttcaagagagaacg

SEQ ID NO: 11 - DNA, 39 bp, Artificial
  oligonucleotide
aattcgttctctcttgaagaacttcagggtcagcttgcg

(19) United States
(12) Patent Application Publication
(10) Pub. No.: US 2008/0312171 A1
(43) Pub. Date: Dec. 18, 2008

(54) Tetracycline-Dependent Regulation of Rna Interference

(75) Inventors: Jacques Mallet, Paris (FR); Roland Vogel, Rueil Malmaison (FR); Lahouari Amar, Villejuif (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignees: CENTRE NATIONAL DE LA RECHERCHE SCIENTIFIQUE, PARIS CEDEX (FR); UNIVERSITE PIERRE ET MARIE CURIE (PARIS VI), PARIS CEDEX (FR)

(21) Appl. No.: 11/915,547

(22) PCT Filed: May 31, 2006
(86) PCT No.: PCT/IB2006/002315

§ 371 Date: Nov. 26, 2007

Related U.S. Application Data
(60) Provisional application No. 60/685,483, filed on May 31, 2005.

(51) Int. Cl. A61K 31/7088; C07H 21/04; C12N 15/00; A61P 43/00; C12N 5/06

(52) U.S. Cl. 514/44; 536/24.1; 435/375; 435/320.1

(57) ABSTRACT

The present invention relates to a tetracycline dependent gene regulatory system or composition controlling the expression of a target gene in a cell and to methods using said system or composition. The present invention more specifically discloses compositions, vectors and methods allowing tetracycline-controlled expression of short-hairpin RNAs (shRNAs), and demonstrates inducible, reversible and stable RNA interference (RNAi) using the same in a cell. The invention can be used to cause reversible control of the expression of any gene and may therefore find applications in the fields of mammalian, in particular human, genetics and molecular therapeutics, in cell and gene therapy, research as well as in genetic studies using transgenic animals.

### FIELD OF THE INVENTION

The present invention relates to a tetracycline dependent gene regulatory system or composition controlling the expression of a target gene in a cell and to methods using said system or composition. The present invention more specifically discloses compositions, vectors and methods allowing tetracycline-controlled expression of short-hairpin RNAs (shRNAs), and demonstrates inducible, reversible and stable RNA interference (RNAi) using the same in a cell. The invention can be used to cause reversible control of the expression of any gene and may therefore find applications in the fields of mammalian, in particular human, genetics and molecular therapeutics, in cell and gene therapy, research as well as in genetic studies using transgenic animals.

### BACKGROUND

The efficient and specific suppression of genes is a prerequisite to study the function of individual genes. RNAi-based gene silencing may be induced by the expression of shRNAs yielding small inhibitory RNAs (siRNAs) after in situ cleavage¹. The method does not require the time-consuming genetic manipulations needed for classical gene knock-out strategies and has therefore emerged as a valuable tool in molecular genetics that may also be applied to human therapy. Since long poly A tails compromise the silencing effect of shRNAs², their expression is appropriately driven by RNA polymerase III which recognizes a run of T residues as a stop signal and does not therefore require a poly A sequence to terminate transcription. In consequence, RNA polymerase III promoters, such as the H1 promoter^{3,4} or the U6 promoter⁵⁻⁷, are widely used to drive the production of shRNAs. Both the H1 promoter and the U6 promoter are constitutively active, and therefore shRNAs can be expressed in a large variety of cells in order to study the consequences of the stable inhibition of target genes. The sequence-specific silencing of target genes by constitutively expressing short-hairpin RNAs¹⁻⁷ allows studies of the consequences of stable gene suppression but is however inappropriate for the analysis of genes essential for cell survival, cell cycle regulation and cell development, for example in the context of transgenic “knock-down” animals. Such studies require conditional gene silencing induced by administration or withdrawal of a small inducer molecule. Conditional suppression of genes is also important for therapeutic applications by permitting to terminate treatments at the onset of unwanted side effects.

Reactivation of a minimal U6 promoter by the Oct-2^{Q}(Q→A) domain was recently employed to establish conditional RNAi by indirectly regulated expression of shRNAs¹⁵: The system was inducible due to ecdysone-regulated expression of the Gal-4-Oct-2^{Q}(Q→A) transcription factor activating a minimal U6 promoter by constitutive binding⁸. Regulation via conditional expression of a target-specific transcription factor however requires additional components.

Another approach of the prior art is based on a Krab-Tet repressor fusion protein²¹, which can conditionally suppress both RNA polymerase II and RNA polymerase III promoters within 3 kb of its binding site²². Expression of the fusion protein allowed conditional RNAi by Dox-controlled inhibition of the expression of shRNAs from a H1 promoter juxtaposed with Tet-operon sequences²³. Nevertheless, the use of this regulatory system may be limited by secondary effects caused by the long-range inhibitory activity of Krab on promoters close to the integration site of the vector.

There have been several attempts to establish conditional RNAi by Dox-regulated steric interference with the formation of the transcription initiation complex at RNA polymerase III promoters¹⁶⁻²⁰. Nevertheless, it is still unclear, where and how many Tet-operons have to be integrated into the promoters to control RNAi effectively.

### SUMMARY OF THE INVENTION

The present invention discloses novel compositions and methods allowing efficient and reversible gene silencing. More particularly, the inventors have developed a regulatory system that allows tetracycline-controlled RNAi. This system is based on a recombinant transactivator that induces transcription of shRNAs from a recombinant promoter, preferably a recombinant RNA polymerase III promoter, in the presence of tetracycline or a derivative thereof. The invention may be implemented using a single transcription factor, thereby facilitating the delivery of conditional RNAi by gene transfer. Furthermore, the present invention may effectively reduce gene expression without causing secondary effects, due to the specificity of the transactivation domain.

Accordingly, the present invention provides a tetracycline dependent gene regulatory system or composition controlling the expression of a target gene in a cell, wherein said system or composition comprises a transactivator induced promoter that modulates RNA interference and preferably said transactivator which is a tetracycline-dependent transactivator. A preferred transactivator according to the present invention is the rtTA-Oct.2 transactivator. Another preferred transactivator according to the present invention is the rtTA-Oct.3 transactivator. Both are described below in the detailed description of the invention.

In one embodiment, the invention provides a gene regulatory system or composition for controlling the expression of a target gene in a cell, wherein said system or composition comprises two expression cassettes, the first cassette comprising a transactivator induced promoter comprising a plurality of transactivator binding sequences operatively linked to a coding sequence producing shRNAs, said shRNA being designed to silence the expression of the target gene, and the second cassette comprising a promoter operatively linked to a sequence encoding a tetracycline-dependent transactivator binding said transactivator binding sequences.

The present invention is further directed to a method for modulating, preferably repressing, expression of a target gene, comprising contacting a cell with a gene regulatory system or composition as disclosed above, said contacting resulting in a modulated, preferably reduced, expression of said target gene depending on the presence or absence of tetracycline or an analog thereof. Advantageously, when said contacting results in a reduced expression of said target gene in the presence of tetracycline or an analog thereof, said repression is reversed upon withdrawal of tetracycline or upon interruption of tetracycline treatment. In a further embodiment of the present invention, when said contacting results in a reduced expression of said target gene in the absence of tetracycline or an analog thereof, said repression is reversed upon administration, adjunction or application of tetracycline or an analog thereof.

The present invention is also directed to a method for modulating, preferably repressing, expression of a target gene wherein said method comprises two steps consisting in successively contacting a cell with a gene regulatory system or composition as disclosed above and with tetracycline or an analog thereof, and wherein said two steps may be inverted.

In another embodiment, the present invention provides a composition comprising two expression cassettes, the first cassette comprising a transactivator induced promoter, preferably a transactivator induced RNA polymerase III promoter, comprising a plurality of transactivator binding sequences operatively linked to a coding sequence producing shRNAs, said shRNA being designed to silence the expression of a target gene, and the second cassette comprising a promoter operatively linked to a sequence encoding a tetracycline-dependent transactivator binding said transactivator binding sequences.

The present invention further provides a nucleic acid comprising a transactivator induced promoter, preferably a transactivator induced RNA polymerase III promoter, comprising a plurality of tetracycline-dependent transactivator binding sequences operatively linked to a coding sequence producing shRNAs. It also provides a vector comprising such a nucleic acid and, optionally, a promoter operatively linked to a sequence encoding a tetracycline-dependent transactivator binding said transactivator binding sequences.

The present invention further provides a composition comprising a vector as described above. In an other embodiment, the present invention provides a vector comprising a nucleic acid comprising a transactivator induced promoter as described above comprising a plurality of transactivator binding sequences operatively linked to a coding sequence producing shRNAs and a second vector comprising a promoter operatively linked to a sequence encoding a tetracycline-dependent transactivator binding said transactivator binding sequences.

The invention can be used to regulate gene expression in cells in vitro, ex vivo or in vivo (e.g., in tissue, organs, etc.). In vitro or ex vivo, the invention may be used as a time and/or dosage-dependent gene regulatory system, in particular in gene function studies, in biocatalysis, in bioprocessing of therapeutic or other molecules, in transgenic plants and animals (for example conditional “knock-down animals”), in high throughput screening applications, in functional genomics and target validation. The invention can also be used for ex vivo and in vivo cell and/or gene animal, preferably human, therapies.

### DETAILED DESCRIPTION OF THE DRAWINGS

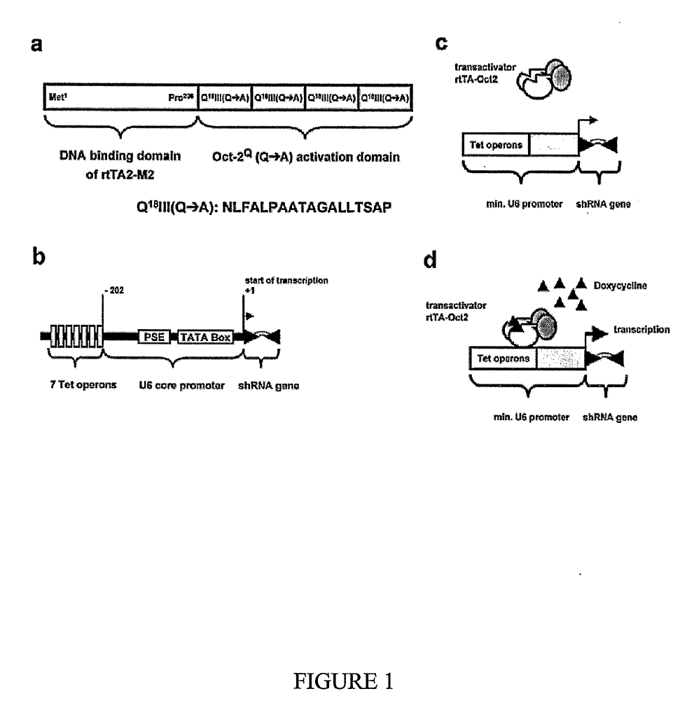

FIG. 1. Schematic diagrams illustrating the regulatory system allowing Dox-induced RNAi. (a) Primary structure of the transactivator rtTA-Oct2 composed of the conditional DNA-binding domain of rtTA2-M2, and the Oct-2^{Q}(Q™A) domain mediating specific induction of a minimal RNA polymerase III promoter⁸. (b) Structure of the minimal U6 promoter used: The 202 bp sequence upstream from the transcription start site was derived from the human U6 promoter and contains the proximal sequence element (PSE) and the TATA box. Upstream from this sequence, seven Tet operons have been inserted to allow conditional binding of the transactivator. (c) In the absence of Dox (off state), rtTA-Oct2 does not bind to the operons and hence shRNAs are not synthesized. (d) In the presence of Dox (on state), the transactivator binds and thereby activates the expression of shRNAs designed to induce the degradation of the respective target mRNAs.

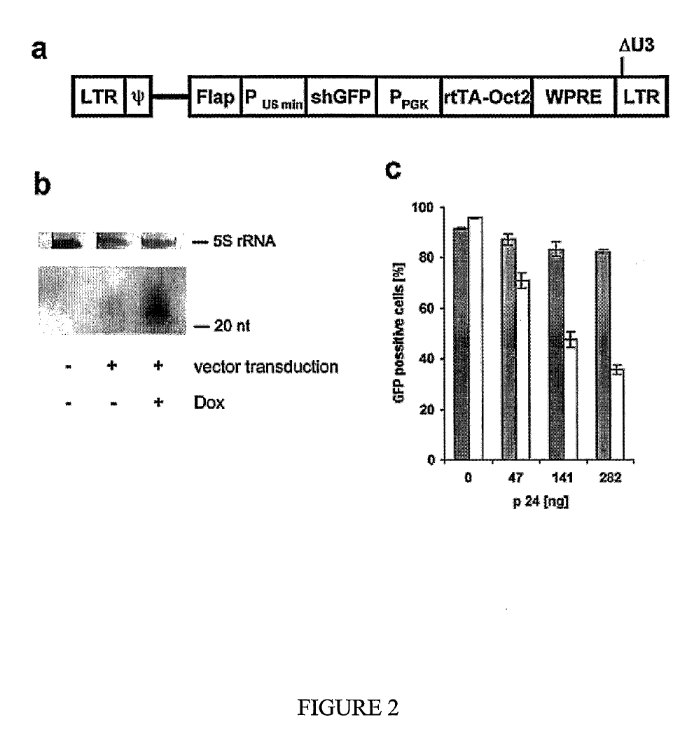

FIG. 2. A single lentiviral vector mediates Dox-regulated RNAi. (a) Design of the vector: LTR, Ψ and Flap are sequences derived from HIV-1 (the long terminal repeats, the packaging sequence and the central Flap element, respectively). P_{U6 min} and P_{PGK} are the Tet-regulated minimal U6 promoter and the phosphoglycerate kinase promoter; WPRE is the Woodchuck hepatitis virus responsive element; rtTA-Oct2, the cDNA encoding the transcription factor rTA-Oct2; and shGFP, the sequence encoding shRNAs designed to silence the expression of GFP. (b) “Northern Blot” analysis of Dox-regulated expression of siRNAs from the vector. HEK 293T GFP cells. (1×10⁵) were incubated for 24 h with and without vector corresponding to 141 ng of protein p24, and cultivated in the presence and absence of 6 μg/ml Dox for 7 days. Then, small RNAs were isolated from the cells and probed for siRNAs designed to silence the expression of GFP. 5S rRNA detected by ethidium bromide staining of the polyacrylamide gel served as an internal control to show equal loading. (c) Experimental validation of RNAi-mediated silencing of GFP. HEK 293T GFP cells (4×10⁴) were incubated overnight with various quantities of vector expressed as ng of protein p24, and cultivated in the absence (grey bars) and in the presence (white bars) of 6 μg/ml Dox for 5 days prior to FACS analysis. Values are averages of percentages of GFP-positive cells ±SD, n=3.

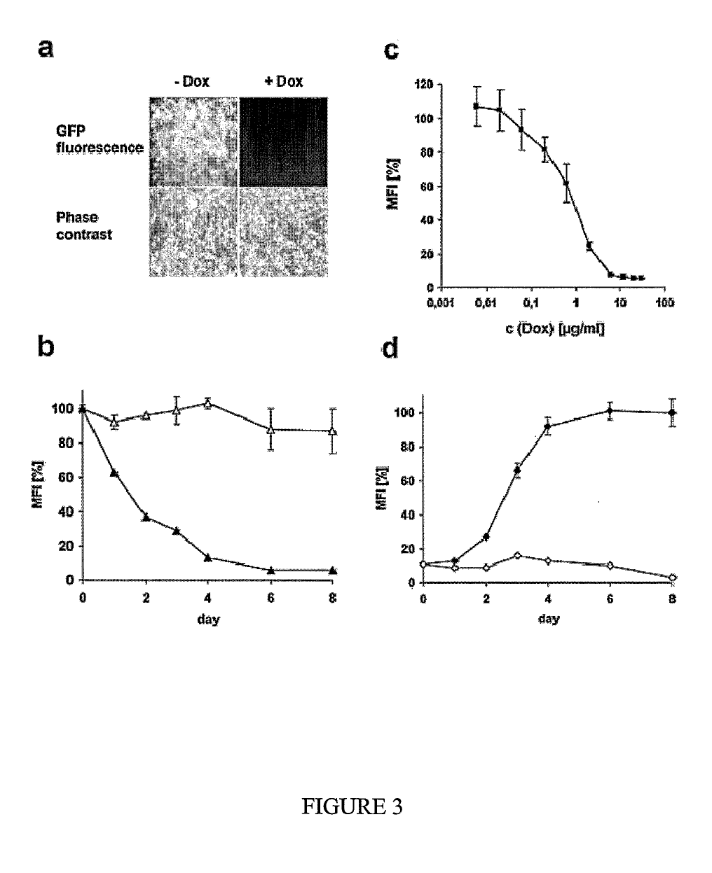

FIG. 3. Characterization of Dox-regulated RNAi in a representative cell clone (C9): (a) Microscopic analysis of cells incubated in the presence or in the absence of 6 μg/ml Dox at 72 h after induction. (b) Time course of Dox-induced RNAi: RNAi was induced or not induced at day 0 by administration of 6 μg/ml Dox and mean intensities of GFP fluorescence were measured by FACS analysis at various times after induction. Filled triangles represent intensities of cells incubated with Dox, open triangles give those of untreated cells. The fluorescence intensity observed at day 0 was defined as 100%, values are means±SE, n=3. (c) Mean intensities (±SE, n=3) of GFP fluorescence obtained by FACS analysis of cells cultivated for 5 days in the presence of various concentrations of Dox. The fluorescence intensity in untreated cells was defined as 100%. (d) Reappearance of GFP fluorescence after withdrawal of Dox: Prior to the analysis, cells were cultivated for 5 days in the presence of 6 μg/ml Dox. At day 0, Dox was withdrawn or not withdrawn and the mean fluorescence intensity was followed by FACS analysis. Filled rhomboids represent values from cells that were not treated with Dox from day 0, open rhomboids give values from cells incubated with 6 μg/ml Dox throughout the experiment. The fluorescence intensity measured 8 days after removal of Dox was defined as 100%, values are means±SE, n=3.

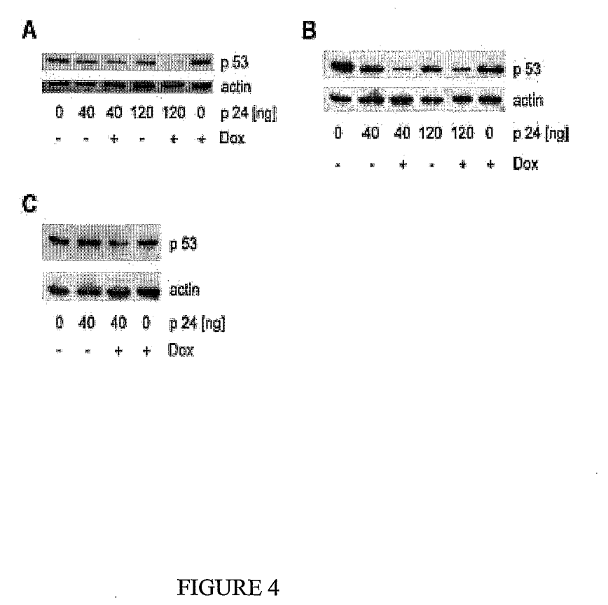

FIG. 4. ‘Western blot’ analysis demonstrating silencing of p53 by Dox-regulated RNAi in (A) HEK 293T cells, (B) MCF-7 cells and (C) A549 cells. Cells (1×10⁵) were incubated overnight with indicated quantities of vector, expressed as ng of protein p24, and then cultivated in the absence and in the presence of 6 μg/ml Dox. After a 5 day (MCF-7 and A549 cells) and a 7 day cultivation (HEK 293T cells), protein was extracted from the cells and analyzed by immunoblotting. Both p53 and actin were detected; the latter served as a control to demonstrate equal loading.

### DETAILED DESCRIPTION OF THE INVENTION

Conditional RNAi can be obtained, in the context of the present invention, by expression of shRNAs from a modified promoter, preferably a modified RNA polymerase III promoter, allowing external control of its activity. Activation of the promoter by a heterologous transcription factor is a key step towards drug-induced transcriptional activity.

The present invention provides a highly efficient and regulated gene expression system including a promoter and a transactivator. Also provided are methods for inducing expression of a nucleic acid using the regulated gene expression system.

In a first embodiment, the present invention relates to a tetracycline dependent gene regulatory system or composition controlling the expression of a target gene in a cell, preferably in a mammalian cell, wherein said system or composition comprises a transactivator induced promoter that modulates RNA interference and preferably said transactivator which is a tetracycline-dependent transactivator.

While exemplified herein with regard to a minimal U6 promoter and to the rtTA-Oct.2 transactivator, the present invention is based on the broader discovery of a gene regulatory system or composition controlling the expression of a target gene in a cell, preferably a mammalian cell, through its ability to modulate the production of shRNA in response to exposure to tetracycline or an analog thereof, wherein said system or composition is comprised of two expression cassettes, the first cassette comprising a transactivator induced promoter comprising a plurality of transactivator binding sequences operatively linked to a coding sequence producing shRNAs, said shRNA being designed to silence the expression of the target gene, and the second cassette comprising a promoter operatively linked to a sequence encoding a tetracycline-dependent transactivator binding said transactivator binding sequences.

Expression cassettes, as used in the present invention, are preferably selected from DNA (in particular cDNA) or RNA, preferably double stranding DNA.

A coding sequence, as mentioned above in the context of the first cassette, is a sequence that encodes at least one functional short-hairpin RNA (shRNA) designed to silence the expression of a target gene. The shRNA is processed within the target cell yielding a small inhibitory RNA (siRNA). This siRNA mediates the specific degradation of the target mRNA by activation of a cellular nuclease. Expression of the coding sequence is controlled by treating the cell with tetracycline or an analogue thereof.

Tetracycline analogs or derivatives thereof may be as useful, or more useful than tetracycline for the purpose of binding the transactivator. As used herein, doxycycline may be preferred to tetracycline in its use in binding to a transactivator. Other useful pharmaceutically acceptable tetracycline analogs include: chlortetracycline, oxytetracycline, demethylchloro-tetracycline, methacycline, doxycycline and minocycline. Thus, a method is provided for controlling expression of a target gene, preferably of shRNA, including the step of contacting a cell containing a gene regulatory system or composition according to the invention including the transactivator-regulated promoter with one of the above described tetracycline or tetracycline analogs.

A promoter useful in the present invention can comprise a RNA polymerase III promoter that can provide high levels of constitutive expression across a variety of cell types and will be sufficient to direct the transcription of a distally located sequence, which is a sequence linked to the 3′ end of the promoter sequence in a cell.

In the first cassette, the promoter region is an inducible promoter, i.e., a transactivator induced promoter, preferably a transactivator induced RNA polymerase III promoter, that can include control elements for the enhancement or repression of transcription of the coding sequence, preferably of the shRNA coding sequence, and can be modified as desired by the user and depending on the context.

A control element is a nucleotide sequence that controls expression of a coding sequence, alone, or in combination with other nucleotide sequences or trans factors. Control elements include, without limitation, operators, enhancers and promoters.

The first cassette described herein typically contains a promoter operatively linked to the transactivator binding sequences to form a regulatable or inducible promoter. Broadly defined, a “promoter” is a DNA sequence that determines the site of transcription initiation for an RNA polymerase. An inducible promoter, in the context of the present invention, is transcriptionally active when bound to a transcriptional activator, which in turn is activated under a specific set of conditions, for example, in the presence or in the absence of a particular combination of chemical signals that affect binding of the transcriptional activator to the inducible promoter and/or affect function of the transcriptional activator itself. Thus, in a first embodiment of the present invention, an inducible promoter is a promoter that, in the absence of the tetracycline inducer or of an analog thereof, does not direct expression, or directs low levels of expression, of a nucleic acid sequence to which the inducible promoter is operatively linked, i.e., the shRNAs encoding sequences. In the presence of tetracycline or an analog thereof, said inducible promoter is activated and directs transcription at an increased level. In a second embodiment of the present invention, an inducible promoter is a promoter that, in the presence of the tetracycline inducer or of an analog thereof, does not direct expression, or directs low levels of expression, of a nucleic acid sequence to which the inducible promoter is operatively linked, i.e., the shRNAs encoding sequences. In the absence of tetracycline or an analog thereof, said later inducible promoter is activated and directs transcription at an increased level.

Suitable promoters for use in the first cassette include, for example, RNA polymerase (pol) III promoters including, but not limited to, the (human and murine) U6 promoters, the (human and murine) H1 promoters, and the (human and murine) 7SK promoters. In addition, a hybrid promoter also can be prepared that contains elements derived from, for example, distinct types of RNA polymerase (pol) III promoters. Modified promoters that contain sequence elements derived from two or more naturally occurring promoter sequences can be combined by the skilled person to effect transcription under a desired set of conditions or in a specific context.

A promoter that is particularly useful in the context of the present invention is compatible with mammalian genes and, further, can be compatible with expression of genes from a wide variety of species. For example, a promoter useful for practicing the invention is preferably a eukaryotic RNA polymerase pol III promoter. The RNA polymerase III promoters have a transcription machinery that is compatible with a wide variety of species, a high basal transcription rate and recognize termination sites with a high level of accuracy. For example, the human and murine U6 RNA polymerase (pol) III and Hi RNA pol III promoters are well characterized and useful for practicing the invention. One skilled in the art will be able to select and/or modify the promoter that is most effective for the desired application and cell type so as to optimize modulation of the expression of one or more genes.

Thus, promoters that are useful in the invention include those promoters that are inducible by the tetracycline external signal or agent or by an analog thereof. A promoter usable in the context of the present invention is selected to be responsive to transcriptional regulation by a transactivator which binds in the presence or absence of tetracycline to the transactivator binding sequences operatively linked to said promoter. The promoter sequence can be one that does not occur in nature, so long as it functions in an eukaryotic cell, preferably a mammalian cell.

In a preferred embodiment of the present invention, the transactivator induced promoter is a recombinant U6, H1 or 7SK promoter, preferably a recombinant U6 or H1 promoter, even more preferably a recombinant human U6 or H1 promoter.

In a preferred gene regulatory system or composition according to the invention, the recombinant U6 promoter is thus a recombinant U6 promoter, preferably human U6 promoter, comprising or linked to a plurality of transactivator binding sequences. Preferably, said transactivator binding sequences replace the functional recognition sites for Staf and Oct-1 in the distal sequence element (DSE) of the U6 promoter, preferably the human U6 promoter.

In a particular embodiment of the invention, the first cassette of the gene regulatory system or composition comprises a plurality of transactivator binding sequences. Said binding sequences preferably comprise from two to ten, preferably from five to nine, even more preferably seven Tet-operon sequences (Tet-operon sequence: CGAGTTTACCACTCCCTATCAGTGATAGAGAAAAGTGAAAGT). Preferably, said Tet-operon sequences are in tandem. Each adjacent Tet-operon sequences may be spaced from each other the same distance in the same nucleic acid sequence. The distance between the two or more Tet-operon adjacent sequences may also vary and/or may be modified to achieve a desired degree of regulation efficiency, that is to vary the maximal and basal transcription rates.

In the second cassette of the system or composition according to the invention, the promoter region is a DNA sequence operatively linked to and modulating the expression of a tetracycline-dependent transactivator, said transactivator binding the transactivator binding sequences of the first cassette.

Suitable promoters for use in the second cassette include, for example, constitutive, regulated, tissue-specific or ubiquitous promoters, which may be of cellular, viral or synthetic origin, such as CMV, RSV, PGK, EF1α, NSE, synapsin, β-actin, GFAP, etc.

As used herein, the term “operatively linked” means that the elements are connected in a manner such that each element can serve its intended function and the elements, together can serve their intended function. In reference to elements that regulate gene expression, “operatively linked” means that a first regulatory element or coding sequence in a nucleotide sequence is located and oriented in relation to a second regulatory element or coding sequence in the same nucleic acid so that the first regulatory element or coding sequence operates in its intended manner in relation with the second regulatory element or coding sequence. In relation to the present invention, a Tet-Operon sequence is operatively linked to a promoter to form a sequence that, when incorporated into a complete gene, including operatively linked Tet-Operon sequences, a promoter and a coding sequence, can be used to control expression of the coding sequence in the presence of a transactivator. A promoter is operatively linked to a coding sequence to promote transcription of that coding sequence.

A preferred transactivator usable in the context of the present invention is a tetracycline-dependent transactivator, preferably the rtTA-Oct2 transactivator composed of the DNA binding domain of rtTA2-M2 and of the Oct-2^{Q}(Q→A) activation domain. Other transactivators may be derived from the Tet repressor protein from E. coli. They may for example comprise all or part of the DNA binding domain of the Tet repressor protein from E. coli. The Tet repressor protein is activated in the absence of tetracycline or an analog thereof. Other transactivators may also for example comprise all or part of the DNA binding domain of rtTA2-M2. Other transactivators may further be chosen from fusion proteins that comprise a DNA binding domain as described above and a transactivation domain which may be chosen for example from the Oct-2^{Q}(Q→A), the p53, the CTF^{p} and the Sp1^{Q} transactivation domains. However, the Oct-2^{Q}(Q→A) activation domain is preferably used to achieve strong activation of the inducible promoter, preferably of the inducible RNA polymerase III promoter, and to avoid side effects due to transactivation of RNA polymerase II promoters in the vicinity of the site where the genome of the vector is integrated into the DNA of the target cell.

As illustrated in the experimental part, activation of the promoter by a heterologous transcription factor may be achieved in case of the U6 promoter by modification of its distal sequence element (DSE) containing binding sites for the transcription factors Staf1 and Oct1. A minimal U6 promoter construct⁸ in which DSE had been replaced by binding sites for the transactivator Gal-4 from yeast, revealed constitutive transcriptional activity when induced by an engineered transcription factor comprising the DNA binding unit of Gal-4 and an artificial transactivation domain referred to as the Oct-2^{Q}(Q→A) domain. This transactivation domain is composed of four copies of the peptide sequence Q¹⁸III(Q→A) comprising the amino acid residues 143 to 160 of the human transcription factor Oct-2 (gene bank accession number: M36653), in which all glutamine residues have been changed to alanine. As the DNA binding domains of Gal-4 and of the tetracycline-dependent transactivator rtTA2-M2⁹ are of similar size, inventors investigated, whether the Oct-2^{Q}(Q→A) domain may be conditionally and functionally linked to a minimal U6 promoter by taking advantage of the Doxycycline (Dox)-dependent interaction of the DNA binding domain of rtTA2M2 with Tet-operon sequences.

As illustrated in the experimental part, Inventors replaced the three minimal VP 16-derived activation domains¹⁰ in rtTA2-M2 by the Oct-2^{Q}(Q→A) domain (FIG. 1A). For conditional binding to an inducible minimal U6 promoter the functional recognition sites for Staf and Oct-1 within the human U6 promoter¹¹ were replaced, in this particular example, by seven Tet-operon sequences (FIG. 1B). The modified promoter and the engineered transcription factor together constitute an advantageous regulatory system allowing conditional RNAi by Dox-dependent expression of shRNAs (FIG. 1C,D).

In a further embodiment, the present invention provides a composition comprising two expression cassettes as described above, the first cassette comprising a transactivator induced promoter comprising a plurality of transactivator binding sequences operatively linked to a coding sequence producing shRNAs, said shRNA being designed to silence the expression of a target gene, and the second cassette comprising a promoter operatively linked to a sequence encoding a tetracycline-dependent transactivator binding said transactivator binding sequences.

The present invention further provides a nucleic acid comprising a transactivator induced promoter, preferably a transactivator induced RNA polymerase III promoter, comprising a plurality of tetracycline-dependent transactivator binding domains operatively linked to a coding sequence producing shRNAs.

Preferred tetracycline-dependent transactivators according to the invention may be chosen from the rtTA-Oct. 2 transactivator composed of the DNA binding domain of rtTA2-M2 and of the Oct-2^{Q}(Q→A) activation domain and the rtTA-Oct. 3 transactivator composed of the DNA binding domain of the Tet-repressor protein (E. coli) and of the Oct-2^{Q}(Q→A) activation domain.

Because the activities of the promoters previously mentioned, such as the U6 and H1 promoters, as well as the localization of expressed nucleic acid sequences can vary from cell type to cell type, if desired, vectors, preferably lentiviral vectors, can be prepared and targeted to the desired targeted cells for modulation of the expression of one or more genes in said targeted cells. The present invention thus also provides a vector comprising a nucleic acid as described above and, optionally, a promoter operatively linked to a sequence encoding a tetracycline-dependent transactivator binding said transactivator binding sequences.

As used herein, the term “vector” refers to one or more nucleic acid molecules capable of transporting another nucleic acid sequence, for example, a ribonucleic acid sequence encompassing a first and second nucleic acid sequence, to which it has been linked. The term is intended to include any vehicle for delivery of a nucleic acid, for example, a virus, plasmid, cosmid or transposon. It is understood that the present invention can be practiced with a variety of delivery vector systems known in the art and able to introduce relatively high levels of nucleic acid sequences into a variety of cells. Suitable viral vectors include yet are not limited to retrovirus, adenovirus and adeno-associated virus vectors.

The term also encompasses vector systems of one or more physically separate vectors, for example, third-generation, retroviral vector systems where the nucleic acid sequences encoding polypeptides having virus packaging functions necessary for generation of a retroviral vector of the invention can be divided onto separate expression plasmids that are independently transfected into the packaging cells.

A viral vector useful for practicing the invention methods, in particular, the therapeutic and prophylactic applications, can thus be derived from a retrovirus. Retroviridae encompass a large family of RNA viruses that is, in part, characterized by its replicative strategy, which includes as essential steps reverse transcription of the virion RNA into linear double-stranded DNA and the subsequent integration of this DNA into the genome of the cell. In a preferred method according to the invention, the vector is a viral vector, preferably a retroviral vector, even more preferably a retroviral vector derived from a lentivirus. A retroviral vector useful in the invention can be a modified lentivirus, for example, an HIV-1, that is used to introduce a nucleic acid sequence into a cell.

A WPRE may be added to the gene regulatory system or composition to enhance the expression of the transactivator used and to stabilize the RNA genome of the vector when a retrovirus vector is used. A flap sequence may further be added to improve transduction of non dividing cells.

The present invention further provides a composition comprising a vector as described above. In an other embodiment, the present invention provides a vector comprising a nucleic acid comprising a transactivator induced promoter comprising a plurality of transactivator binding sequences operatively linked to a coding sequence producing shRNAs and a second vector comprising a promoter operatively linked to a sequence encoding a tetracycline-dependent transactivator binding said transactivator binding sequences.

The present invention further relates to a method for modulating, preferably repressing, expression of a target gene, comprising contacting a cell with a gene regulatory system or composition according to the invention said contacting resulting in a modulated, preferably reduced, expression of said gene in the presence or absence of tetracycline or an analog thereof depending, as explained previously, on the transactivator used.

Invention also relates to a method for repressing expression of a target gene, wherein said method comprises two steps consisting in successively contacting a cell with an inventive gene regulatory system or composition as described previously and with tetracycline or an analog thereof, and wherein said two steps may be inverted.

The target gene expression repression can be reversed upon withdrawal of tetracycline or upon interruption of tetracycline treatment or on the contrary upon administration, adjunction or application of tetracycline or an analog thereof, depending, as explained previously, on the transactivator used. Such a method can be realized in a dose- and time-dependent manner.

Quantitation of gene expression or repression in a cell can be measured by measure of a gene product produced by the modulated gene as well as, indirectly, by measuring phenotypic changes associated with expression or repression of the gene product. For example, the amount of gene product in the cell can be detected with a hybridization probe having a nucleotide sequence, or translated polypeptide can be detected with an antibody raised against a polypeptide epitope. In addition, a phenotypic change associated with expression or repression of the gene can be measured, for example, cell type differentiation.

The one or more target gene whose expression may be modulated can be any gene. In particular genes that are essential for cell survival, cell cycle regulation and/or cell development may be modulated such as, for example, oncogenes and genes involved in apoptosis and neurodegeneration.

In a particular embodiment of the invention, the gene the expression of which is modulated, preferably repressed, is specific to expression in the nervous system, preferably in the nervous system of a mammal, even more preferably of a human.

In a method according to the invention, the gene regulatory system or composition may be contacted or incubated with or may be administered or delivered to a cell in vitro, in vivo or ex vivo.

As used herein, the term “in vitro” means an environment outside of a living organism. Applications performed using whole-cell or fractionated extracts derived from lysed cells, or performed with reconstituted systems, are encompassed within the term “in vitro” as used herein. Furthermore, both living cells derived from an organism and used directly (primary cells) as well as cells grown for multiple generations or indefinitely in culture are encompassed within the term “in vitro” as used herein. A target cell may be an eukaryotic cell, preferably a mammalian cell, such as a mammalian fertilized oocyte, a mammalian embryonic or neuronal stem cell, even more preferably a human, a murine, porcine or bovine cell.

As used herein, the term “in vivo” means an environment within a living organism. Such a living organism can be, for example, a multi-cellular organism such as a rodent, mammal, primate or human or another animal such as an insect, worm, frog or fish, or a unicellular organism such as a single-celled protozoan, bacterium or yeast. The cell can be in an in utero animal, or in an ex utero animal. In vivo applications of the invention include applications in which a gene regulatory system or composition of the invention is introduced, for example, into cells within a living mammal, preferably a human being, within a living animal or a plant.

As used herein, the term “ex vivo” means that the invention is introduced into living cells “in vitro” and that the manipulated cells are subsequently implanted into a living mammal, preferably a human being, within a living animal or a plant.

In a particular embodiment, at least one or at least two distinct vectors as described above are used, in a method according to the invention, to deliver the inventive gene regulatory system or composition to the cell and may be administered simultaneously or sequentially.

Further aspects and advantages of the present invention will be disclosed in the following examples, which should be regarded as illustrative and not limiting the scope of the present application.

### EXAMPLES

As a proof-of-principle, tetracycline-controlled RNAi was used to regulate the expression of GFP in HEK 293T cells stably expressing this transgene. In the presence of doxycycline, GFP was down-regulated by RNAi in a dose- and time-dependent manner. In particular, silencing of GFP was reversible after withdrawal of doxycycline, as was followed by the reappearance of GFP fluorescence.

As a delivery system, inventors constructed a single lentivirus vector by inserting two expression cassettes into its backbone (FIG. 2A). The first cassette contained the minimal U6 promoter and was used to produce shRNAs designed to silence the expression of GFP as described⁴. The second cassette was employed to express the engineered transcription factor rtTA2-Oct2 composed of the DNA binding domain of rtTA2-M2 and the Oct-2^{Q}(Q→A) activation domain. The transcription factor was constitutively transcribed from the phosphoglycerate kinase (PGK) promoter; and the polyA sequence of the vector in the 3′ long terminal repeat (LTR) was used for polyadenylation. The vector contained a WPRE sequence¹² to enhance the expression of rtTA2-Oct2 and to stabilize the RNA genome of the vector during the production of vector particles in transiently transfected HEK 293T cells. A Flap sequence was also included to improve transduction of non-dividing cells¹³. For safety reasons the U3 promoter region was deleted from the 3′ LTR so that the vector was self-inactivating¹⁴.

A HEK 293T GFP cell-clone that stably expresses GFP as a transgene was transduced with the vector construct. Cells were cultivated in the presence and absence of Dox (6 μg/ml), before small RNAs were isolated from the cultures as well as from controls (non-transduced HEK 293T-GFP cells). “Northern Blot”. analysis of the RNA samples revealed that siRNAs designed to silence GFP were expressed in transduced cells cultivated in the presence of Dox (FIG. 2B). The siRNAs were not detected in non-transduced cells. In transduced cells cultivated without Dox no signal exceeding the detection threshold was observed. “Northern Blotting” did not allow detection of shRNAs probably because of their rapid cleavage into siRNAs by Dicer nuclease.

Subsequently, HEK 293T GFP cells were transduced with various amounts of vector and incubated in the presence and absence of Dox (6 μg/ml). Incubation with Dox reduced the number of GFP-expressing cells by up to 60% as was determined by FACS analysis (FIG. 2C). The decrease in GFP-positive cells correlated with the amount of vector applied. The number of GFP positive cells among transduced cells incubated in the absence of Dox was 10-15% lower than among non-transduced cells. This difference also correlated with the amount of vector applied and may have been caused by leakage expression of shRNAs in cells containing multiple copies of the vector genome.

To establish uniform conditions for precise characterization of the regulatory system, cell clones were amplified from individual transduced cells. Several clones were obtained that displayed Dox regulated expression of GFP (see supplementary table). Fluorescence microscopy of a representative clone (C9) demonstrated that GFP was only expressed in the absence of Dox (FIG. 3A). Inventors then used FACS analysis to study the effect of Dox on the expression of GFP. The addition of Dox to the cells was followed by a significant decrease in GFP fluorescence within 24 h; after 5-6 days the reduction of GFP fluorescence was 90% (FIG. 3B). In the absence of Dox there were no changes in GFP fluorescence during the incubation. To determine the minimal concentration of Dox required to induce RNAi, cells of the clone C9 were incubated with various concentrations of Dox (FIG. 3C). A concentration of about 6 μg/ml was required to induce a 90% suppression of GFP within 5 days. Lower concentrations of Dox were either ineffective or caused incomplete or delayed RNAi. To test inducible RNAi for reversibility, cells of the clone C9 were cultivated for 5 days in the presence of Dox. Then, Dox was removed and the expression of GFP was followed. GFP fluorescence had increased significantly 48 h after the removal of Dox (FIG. 3D), although incubation without Dox for 5-6 days was required to restore maximal expression of GFP. No increase in GFP fluorescence was detected in cells incubated with Dox throughout the experiment.

In a next step inventors used the regulation system according to the invention for the silencing of the p53 gene.

This gene was chosen because of detectable expression in mammalian cells, availability of reliable antibodies to monitor levels of the protein, and the existence of an efficient shRNA. A recent study (25) showed that genetic deletion of p53 suppressed neurodegeneration in animal models of Huntington's disease. Local and regulated downregulation of p53 thus constitute a novel gene therapy approach for the treatment of Huntington disease patients.

Inventors constructed a second vector, which contained a shRNA encoding sequence designed to silence expression of human p53 as described (1). HEK 293T cells, MCF-7 cells and A549 cells were transduced with various amounts of vector and incubated in the presence and absence of Dox (6 μg/ml) for 5-7 days before protein was extracted from the cultures as well as from non-transduced controls. ‘Western blot’ analysis of protein samples containing identical amounts of protein revealed that p53 levels were efficiently reduced when transduced cells were incubated in the presence of Dox (FIG. 4). An up to 90% inhibition of the expression of p53 was observed in Dox treated cultures of transduced cells as assessed by densitometric analysis of the Blot data. No down-regulation of p53 was observed, or at best some minimal silencing because of leakage expression of shRNAs, was obtained when transduced cells were cultivated in the absence of Dox. The expression of p53 was not reduced when non-transduced cells were incubated in the presence of Dox (6 μg/ml).

Considered together, inventors findings indicate that the engineered minimal U6 promoter was conditionally reactivated by Dox-controlled binding of rtTA2-Oct2 containing the Oct-2^{Q}(Q→A) domain for transactivation. The minimal U6 promoter and the recombinant transcription factor together formed a regulatory system allowing conditional RNAi by Dox-controlled production of shRNAs.

### Methods

### Plasmid Constructions

The plasmids pUHR 10-3 and pUHRT 62-1, which contain the components of the Tet regulatory system, were kindly provided by H. Bujard (Zentrum für Molekulare Biologie, Heidelberg, Germany). The plasmid pcDNA-Δ that allows the use of Bbs I in subsequent cloning experiments was generated by self-ligation of the vector fragment obtained by Pst I digestion of the plasmid pcDNA 3 (Invitrogen, Cergy Pontoise, France). The core unit of the human U6 promoter that did not contain the functional binding sites for the transcription factors Staf and Oct-1¹¹ was amplified by polymerase chain reaction (PCR) from genomic DNA of HEK293T cells. The oligonucleotides 5′-CGACGCGTTGCAGAGCTCGTTAGAGAGATAATTAGAATTAATTTGAC TGTAAACACAAAG-3′, and 5′-CGGGATCCAGAAGACCACGGTGTTTCGTCCTTTCCACA AGAT-3′ (Eurogentec, Angers, France) were the sense and antisense primers respectively, and the DNA fragment amplified contained both a Mlu I and a Sac I site upstream, and a Bbs I and a Bam H I site downstream from the truncated U6 promoter. The fragment was inserted between the Mlu I and Bam H I sites of pcDNA-A yielding the plasmid pcDNA-ΔU6t. A Mlu I-Sac I fragment containing seven Tet operon sequences was amplified by PCR from pUHR 10-3 and inserted between the Mlu I and Bam H I sites of pcDNA-ΔU6t to give pcDNA-ΔU6 min. The DNA fragment encoding shRNAs designed to silence expression of GFP (shGFP) was generated by annealing the oligonucleotides 5′-ACCGCAAGCTGACCCTGAAGTTCTTCAAGAGAGA ACTTCAGGGTCAGCTTGCTTTTTCTCGAGG-3′, and 5′-GATCCC TCGAGAAAAAGCAA GCTGACCCTGAAGTTCTCTCTTGAAGAACTTCAGGGTCAGCTTG-3′ and inserted into pcDNA-ΔU6 min linearized by Bbs I-Bam H I digestion. The resulting plasmid was named pcDNA-ΔU6 min-shGFP.

An Eco R I-Bam H I fragment encoding the DNA binding domain of rtTA2-M2⁹ was amplified by PCR from pUHRT 62-1 using the oligonucleotides 5′-CGGAATTCACCATGTCTAGACTG GACAAGAGCAAAG-3′ and 5′-CGGGATCCTGAAGACTACGGTCCGCCGCTTTCGCACT TTAGCTGT-3′ as the sense and antisense primers, respectively. Upstream from the Bam H I site the fragment contained a stop codon and a Bbs I site allowing extension with a fragment encoding additional amino acid residues. Insertion of the fragment between the Eco R I-Bam H I sites of pcDNA-A yielded the plasmid pcDNA-Δ/rtTA2-M2trunc. The DNA fragment coding the peptide sequence Q¹⁸III(Q→A) was generated by annealing the oligonucleotides 5′-ACCGAAC CTGTTCGCTCTCCCCGCTGCAACAGCGGGAGCCCTACTGACATCAGCACCGTA GTCTTCG-3′ and 5′-GATCCGAAGACTACGGTGCTGATGTCAGTAGGGCTCCCGCTGTTGCAG CGGGGAGAGCGAACAGGTT-3′ and was inserted into pcDNA-Δ/rtTA2-M2trunc linearized by Bbs I-Bam H I digestion. The resulting plasmid contained again a stop codon and a Bbs I site upstream from the Bam H I site allowing further rounds of extension with the same fragment. Extension with the fragment encoding Q¹⁸III(Q→A) was repeated three times yielding the plasmid containing the rtTA2-Oct2 cDNA. The sequence encoding rtTA2-Oct2 was recovered by Eco R I-Bam H I digestion and inserted into pΔ500rtTA2-M2-WPRE²⁴ from which rtTA2-M2 had been removed by Eco R I-Bam H I digestion. A Sal I-Eco R I fragment containing the PGK promoter was amplified by PCR and inserted into the Eco R I-Sal I site upstream from rtTA2-Oct2 yielding pΔ500PGK-rtTA2-Oct2-WPRE.

The cassette allowing shGFP expression was recovered from pcDNA-ΔU6 min-shGFP by Mlu I-Spe I digestion and inserted into the lentivector precursor plasmid pTrip-CMVmin-WPRE²⁴ from which the element CMVmin had been removed by Mlu I-Spe I digestion. The WPRE sequence was removed from the resulting plasmid (pTrip-U6 min-shGFP-WPRE) by Spe I-Kpn I digestion and replaced by the rtTA2-Oct2 expression cassette recovered from pΔ500PGK-rtTA2-Oct2-WPRE by Nhe I-KpnI digestion. The resulting plasmid, pTrip-U6 min-shGFP-PGK-rtTA2-Oct2-WPRE, was used for the production of lentivirus vector particles.

The DNA fragment encoding the riboprobe for the detection of the GFP silencing siRNAs was generated by annealing the oligonucleotides 5′-GATCCGCAAGCTGACCCTGAAGTTCTTCA AGAGAGAACG-3′ and 5′-AATTCGTTCTCTCTTGAAGAACTTCAGGGTCAGCTTGCG-3′ and was inserted between the Bam H I-Eco R I sites of pcDNA 3. All plasmid constructs were verified by sequencing using a ABI-PRISM 13100 DNA sequencer (Applied Biosystems, Courtabeuf, France)

### Cell Culture, Lentiviral Transductions and Selection of Transduced Cells

All cell clones derived from HEK 293T cells were cultivated at 37° C. under a humidified atmosphere of 5% CO2/95% air in DMEM supplemented with 10% fetal calf serum, 20 units/ml penicillin G and 20 μg/ml streptomycin sulfate. Lentivirus vector particles were produced by transient cotransfection of HEK 293T cells by the vector plasmid, an encapsidation plasmid (p 8.7), and a VSV expression plasmid (pHCMV-G) as described¹³. Vector stocks were tittered by determination of the amount of the p24 capsid protein using an HIV-1 core profile enzyme linked immunosorbent assay (Beckman Coulter, Roissy, France). For transduction HEK 293T-GFP cells were incubated overnight with vector in the presence of 10 μg/ml DEAE dextran (Sigma-Aldrich, St. Quentin Fallavier, France). Transduced cells were selected after 5 days of cultivation in the presence of 6 μg/ml Dox using a FACSVantage SE cell-sorting instrument (Becton Dickinson, Rungis, France). Selected clones were expanded and analyzed by fluorescence microscopy and FACS.

### Northern Blot Analysis

A ³²P-labeled riboprobe was transcribed from the plasmid encoding the riboprobe using α-³²P ATP (Amersham Biosciences, Orsay, France) and the Riboprobe System-T7 (Promega, Char-bonnières, France). Small RNAs were isolated from aliquots of 10⁷ cells with the mirVana™ PARIS™ Kit (Ambion, Huntingdon, UK). Samples containing 3.3 μg of small RNAs were denatured by heating at 95° C. for 5 min in the presence of 50% formamide. After electrophoresis on a 15% polyacrylamide gel in the presence of 8 M urea the RNA was stained with ethidium bromide and examined on a transilluminator. The RNA was then transferred by electroblotting to a BrightStar-Plus Nylon membrane (Ambion), fixed by UV crosslinking and hybridized to the probe. The resulting ³²P-labeled RNA-RNA hybrids were detected by autoradiography using Hyperfilm™ MP (Amersham Biosciences).

Supplementary Table: Cell clones showing regulated expression of GFP
mediated by Dox-controlled RNAi

| Clone # | MFI (−Dox) | MFI (+Dox) | Regulation factor |
|---|---|---|---|
| C1 | 210 | 4 | 52 |
| C9 | 310 | 32 | 9.7 |
| D8 | 220 | 9 | 24 |
| H6 | 340 | 77 | 4.4 |

### Western Blot Analysis

Cell extracts were prepared in lysis buffer [25 mM Tris-HCl (pH 7.5), 1% Triton X-100, 0.5% sodium deoxycholate, 5 mM EDTA, 150 mM NaCl] containing a cocktail of protease inhibitors (Roche, Meylan, France). The protein samples (30 μg) were separated on SDS-9% polyacrylamide gels and then transferred to Protan nitrocellulose membranes (Schleicher and Shuell, Dassel, Germany) in an electroblotting apparatus, using standard procedures (26). Immunodetection was performed as described in Tejedor-Real et al. (27), using a monoclonal anti-p53 antibody (BD Biosciences, Erembodegem, Belgium), a monoclonal anti-actin antibody (Chemicon, Hampshire, UK) and an anti-mouse Ig-horseradish peroxidase (HRP) conjugate (Amersham Biosciences).

### REFERENCES

1. Brummelkamp, T. R., Bernards, R. & Agami, R. A system for stable expression of short interfering RNAs in mammalian cells. Science 296, 550-553 (2002).
2. Xia, H., Mao, Q., Paulson, H. L. & Davidson, B. L. siRNA mediated gene silencing in vitro and in vivo. Nat. Biotech. 20, 1006-1010 (2002).
3. Abbas-Terki, T., Blanco-Bose, W., Déglon, N., Pralong, W. & Aebischer, P. Lentiviral-mediated RNA interference. Hum. Gene Ther. 13, 2197-2201 (2002).
4. Tiscornia, G., Singer, O., Ikawa, M. & Verma, I. M. A general method for gene knockdown in mice by using lentiviral vectors expressing small interfering RNA; Proc. Natl. Acad. Sci. USA 100.1844-1848 (2003).
5. Qin, X.-F., An, D. S., Chen, I. S. Y. & Baltimore, D. Inhibiting HIV-1 infection in human T cells by lentiviral mediated delivery of small interfering RNA against CCR5. Proc. Natl. Acad. Sci. USA 100, 183-184 (2003).
6. Rubinson, D. A. et al. A lentivirus-based system to functionally silence genes in primary mammalian cells, stem cells and transgenic mice by RNA interference. Nature Genetics 33, 401-406 (2003).
7. Stewart, S. A. et al. Lentivirus-delivered stable gene silencing by RNAi in primary cells. RNA 9, 493-501 (2003).
8. Das, G., Hinkley, C. S. & Herr, W. Basal promoter elements as a selective determinant of transcriptional activator function. Nature 374, 657-660 (1995).
9. Urlinger, S. et al. Exploring the sequence space for tetracycline-dependent transcriptional activators: Novel mutations yield expanded range and sensitivity. Proc. Natl. Acad. Sci. USA 97, 7963-7968 (2000).
10. Baron, U., Gossen, M. & Bujard, H. Tetracycline-controlled transcription in eukaryotes: novel transactivators with graded transactivation potential. Nucleic Acids Res. 25, 2723-2729 (1997).
11. Danzeiser, D. A., Urso, O. & Kunkel, G. R. Functional characterization of elements in a human U6 small nuclear RNA gene distal control region. Mol. Cell. Biol. 13, 4670-4678 (1993).
12. Zufferey, R., Donello, J. E., Trono, D. & Hope, T. J. Woodchuck hepatitis virus posttranscriptional regulatory element enhances expression of transgenes delivered by retroviral vectors. J. Virol. 73, 2886-2892 (1999).
13. Zennou, V. et al. The HIV-1 DNA flap stimulates HIV vector mediated cell transduction in the brain; Nat. Biotech. 19, 446-450 (2001).
14. Zufferey, R. et al. Self-inactivating lentivirus vector for safe and efficient in vivo gene delivery. J. Virol. 72, 9873-9880 (1998).
15. Gupta, S., Schoer, R. A., Egan, J. E., Hannon, G. J. & Mittal, V. Inducible, reversible, and stable RNA interference in mammalian cells. Proc. Natl. Acad. Sci. USA 101, 1927-1932 (2004).
16. van de Wetering, M. et al. Specific inhibition of gene expression using a stably integrated, inducible small-interfering-RNA vector. EMBO rep. 4, 609-615 (2003).
17. Czauderna, F. et al. Inducible shRNA expression for application in a prostate cancer mouse model. Nucleic Acids Res. 31, e127 (2003).
18. Matsukura, S., Jones P. A. & Takai, D. Establishment of conditional vectors for hairpin siRNA knockdowns. Nucleic Acids Res. 31, e77 (2003).
19. Hosono T. et al. Adenovirus mediated Doxycycline inducible RNA interference Hum. Gene Ther. 15, 813-819 (2004).
20. Lin X. et al. Development of a tightly regulated U6 promoter for shRNA expression. FEBS Lett. 577, 376-380 (2004).
21. Deuschle, U., Meyer, W. K. & Thiesen, H.-J. Tetracycline-reversible silencing of eukaryotic promoters. Mol. Cell. Biol. 15, 1907-1914 (1995).
22. Moosmann, P., Georgiev, O., Thiesen, H.-J., Hagmann, M. & Schaffner, W. Silencing of RNA polymerase II and III-dependent transcription by the Krab protein domain of Kox1, a Krüppel-type zinc finger factor. Biol. Chem. 378, 669-677 (1997).
23. Wizneroowicz, M. & Trono, D. Conditional suppression of cellular genes: Lentivirus vector-mediated drug-inducible RNA interference. J. Virol. 77, 8975-8961 (2003).
24. Vogel, R., Amar, L., Do Thi, A., Saillour, P. & Mallet, J. A single lentivirus vector mediates doxycycline-regulated expression of transgenes in the brain. Hum Gene The. 15, 157-165 (2004).
25. Bae, B.-I., Xu, H., Igarashi, S., Fujimuro, M., Agrawal, N., Taya, Y., Hayward, S. D., Moran, T. H., Montell, C., Ross, C. A. et al. (2005) p53 mediates cellular dysfunction and behavioural abnormalities in Huntington's disease. Neuron, 47, 29-41.
26. Towbin, H., Staehelin, T. and Gordon, J. (1979) Electrophoretic transfer of proteins from polyacrylamide gels to nitrocellulose sheets: procedure and some applications. Biotechnology, 24, 145-149.
27. Tejedor-Real, P., Vogel, R., Mallet, J. and Faucon-Biguet, N. (2005) Gi/Go protein-dependent presynaptic mechanisms are involved in clozapine induced down-regulation of tyrosine hydroxylase in PC 12 cells. J. Neurosci. Res., 81, 739-745.

1-27. (canceled)

28. A tetracycline dependent gene regulatory system or composition controlling the expression of a target gene in a cell, wherein said system or composition comprises a transactivator induced promoter that modulates RNA interference and said transactivator which is a tetracycline-dependent transactivator.

29. The gene regulatory system or composition according to claim 28, wherein the transactivator is the rtTA-Oct.2 transactivator composed of the DNA binding domain of rtTA2-M2 and of the Oct-2^{Q}(Q→A) activation domain.

30. The gene regulatory system or composition according to claim 28, wherein the transactivator is the rtTA-Oct.3 transactivator composed of the DNA binding domain of the Tet-repressor protein (E. coli) and of the Oct-2^{Q}(Q→A) activation domain.

31. The gene regulatory system or composition according to claim 28, wherein the transactivator induced promoter is derived from a recombinant U6, H1 or 7SK promoter.

32. The gene regulatory system or composition according to claim 31, wherein the recombinant U6 promoter is a recombinant U6 promoter comprising a plurality of transactivator binding sequences replacing the functional recognition sites for Staf and Oct-1 in the distal sequence element (DSE) of the U6 promoter.

33. A gene regulatory system or composition for controlling the expression of a target gene in a cell, wherein said system or composition comprises two expression cassettes, the first cassette comprising a transactivator induced promoter comprising a plurality of transactivator binding sequences operatively linked to a coding sequence producing shRNAs, said shRNA being designed to silence the expression of the target gene, and the second cassette comprising a promoter operatively linked to a sequence encoding a tetracycline-dependent transactivator binding said transactivator binding sequences.

34. The gene regulatory system or composition according to claim 33, wherein the plurality of transactivator binding sequences comprises from two to ten Tet-operon sequences.

35. The gene regulatory system or composition according to claim 34, wherein the Tet-operon sequences are in tandem.

36. A method for repressing expression of a target gene in vitro, ex vivo or in vivo, comprising contacting a cell with a gene regulatory system or composition, wherein said system or composition comprises a transactivator induced promoter that modulates RNA interference and said transactivator which is a tetracycline-dependent transactivator, said contacting resulting in a reduced expression of said target gene in the presence or in the absence of tetracycline or an analog thereof, depending on the transactivator used.

37. The method according to claim 36, wherein one vector is used to deliver said gene regulatory system or composition to said cell.

38. The method according to claim 36, wherein at least two distinct vectors, which may be administered simultaneously or sequentially, are used to deliver said gene regulatory system or composition to said cell.

39. The method according to claim 38, wherein said vector is a viral vector.

40. The method according to claim 39, wherein said viral vector is derived from a lentivirus.

41. The method according to claim 36, wherein said repression is reversed upon interruption of tetracycline treatment or upon administration of tetracycline or an analog thereof, depending on the transactivator used.

42. The method according to claim 36, wherein said gene is specific to expression in the nervous system.

43. A method for modulating expression of a target gene in vitro, ex vivo or in vivo, wherein said method comprises two steps consisting in successively contacting a cell with (i) a gene regulatory system or composition, wherein said system or composition comprises a transactivator induced promoter that modulates RNA interference and said transactivator which is a tetracycline-dependent transactivator, and with (ii) tetracycline or an analog thereof, and wherein said two steps may be inverted.

44. A nucleic acid comprising a transactivator induced promoter comprising a plurality of tetracycline-dependent transactivator binding sequences operatively linked to a coding sequence producing shRNAs.

45. A vector comprising a nucleic acid according to claim 44.

46. The vector according to claim 45, further comprising a promoter operatively linked to a sequence encoding a tetracycline-dependent transactivator binding said transactivator binding sequences.
